(19) United States
(12) Patent Application Publication
(10) Pub. No.: US 2003/0166169 A1
(43) Pub. Date: Sep. 4, 2003

(54) Method for constructing viral nucleic acids in a cell-free manner

(76) Inventors: Hal S. Padgett, Vacaville, CA (US); John A. Lindbo, Vacaville, CA (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/196,677

(22) Filed: Jul. 15, 2002

Related U.S. Application Data
(63) Continuation of application No. 09/359,303, filed on Jul. 21, 1999, now abandoned.
Application No. 09/359,303 is a continuation-in-part of application No. 09/232,170, filed on Jan. 15, 1999, now abandoned.
Application No. 09/232,170 is a continuation-in-part of application No. 09/008,186, filed on Jan. 16, 1998.

(51) Int. Cl. C12P 19/34; C12Q 1/70

(52) U.S. Cl. 435/91.2; 435/5

(57) ABSTRACT

The present invention relates to a method for constructing viral nucleic acids in a cell-free manner. In essence, the cell-free method entails the immobilization of a fragment of a double-stranded DNA sequence on a solid support and the assembly of the remaining fragments of the double-stranded DNA sequence onto the immobilized fragment. If the viral nucleic acid is derived from an RNA virus, the instant method further comprises the step of in vitro transcription of the assembled double-stranded DNA sequence to yield an RNA viral nucleic acid.

This application is a continuation application of U.S. application Ser. No. 09/359,303, filed Jul. 21, 1999, which is a continuation-in-part of U.S. patent application Ser. No. 09/232,170, filed Jan. 15, 1999, which is a continuation-in-part of U.S. patent application Ser. No. 09/008,186, filed Jan. 16, 1998.

### FIELD OF THE INVENTION

The present invention relates generally to the field of molecular biology and viral genetics. Specifically, the present invention relates to a method for constructing viral nucleic acids in a cell-free manner.

### BACKGROUND OF THE INVENTION

Recombination at the genetic level is important for generating diversity and adaptive change within the genome of virtually all organisms. Recombinant DNA technology is based upon simple “cut-and-paste” cloning methods for manipulating nucleic acid molecules in vitro. Typically, a DNA fragment of interest and an appropriate vector are first digested with a restriction endonuclease enzyme which recognizes specific sequences within the DNA. The ends of the restriction endonuclease-treated DNA fragment and vector are further manipulated, if necessary, to make them compatible for ligation or joining together. DNA ligase is then added to the mixture, ligating the DNA fragment and the vector together. This genetic assembly containing the DNA sequence of interest, an origin of DNA replication and a selectable gene is then inserted into a living cell, grown up, and positively selected to yield a culture capable of providing high yields of individual recombinant DNA molecules, or their products, such as RNA or protein.

Significant improvements have been made to the recombinant DNA-technology over the last two and half decades. The polymerase chain reaction (PCR) has become one of the most powerful tools in molecular cloning and found utility in many aspects of the modern recombinant DNA technology. Rapid amplification and isolation of specific DNA sequences are routinely achieved using PCR based technologies. If PCR results in a single product or if the desired product can be readily separated from the contaminating products, there is often no need for cloning. However, when PCR products are heterogeneous, cloning is typically required in order to isolate PCR specific products. Cloning can be performed by conventional procedures such as the use of restriction sites present in the PCR products or by blunt end cloning. The blunt end cloning of the PCR products is often inefficient and requires the removal of the 3′ overhang generated by Taq polymerase. As an alternative to the blunt end cloning, restriction enzymes may be introduced into the PCR primers so that the subsequent digestion of the PCR products with restriction enzymes results in fragments ready to be cloned into the specific sites of the vectors. However, when a complex population of DNA molecules, such as that found in a cDNA library, is used as the starting material for cloning and a given restriction endonuclease is used to treat the DNA fragment of interest to render the appropriate termini for ligation to the cloning vector, the recognition sequence for that enzyme may occur with a certain frequency within the population, rendering the DNA molecule bearing that sequence truncated after digestion.

Several restriction enzyme-free and ligation-independent cloning methods have been introduced. In one method, long (10-12 bases) single-stranded regions are generated at the ends of the PCR products and an appropriate vector using T4 DNA polymerase. The protruding ends of the PCR products are annealed specifically to complementary DNA sequences on the vector and subsequently transformed into bacteria competent cells (Aslanidis et al., Nucleic Acids Research 18(20):6069-6074 (1990); and Aslanidis et al., PCR Methods Appl. 4:172-177 (1994)). Another method for generating PCR products with protruding ends utilizes the enzyme uracil DNA glycosylase (UDG) (Rashtchian et al., Anal. Biochem. 206:91-97 (1992). Rashtchian, Curr. Opin. Biotechnol 6(1):30-36 (1995)). This method involves the use of DNA primers that contain a 5′ tail, into which deoxyuridine residues have been incorporated. These primers result in the incorporation of deoxyuridine-containing sequences into the 5′ ends of the PCR products. The selective removal of deoxyuridine residues by UDG generates single-stranded 3′ overhangs in the PCR products, which are then annealed to an appropriate vector with complementary single-stranded ends. This circularized product can then be transformed into bacteria competent cells.

Although these cloning methods circumvent the use of restriction enzymes and ligases, they still utilize bacteria cell culture, such as E. coli, for the selection and production of the desired product. Passage of certain clones, such as plasmid-based viral clones, through E. coli has been observed to result in the instability of the plasmid for a certain proportion of the time. For example, the bacterial cells may simply “screen out” certain viral clones. The cause of this instability is unclear, but may be related to the insert size, sequence or to the toxicity resulting from the gene expression using cryptic promoter sequences.

There remains a need in the art to increase the representation of gene sequences in viral expression libraries by bypassing the genetic bottleneck of propagation in a cell culture. There is also a need for eliminating the use of prokaryotic hosts and for minimizing or avoiding the risks associated with bacteria contamination resulting from the use of bacteria as intermediaries in the cloning process.

In the instant invention, libraries of viral nucleic acid sequence variants are generated in a cell-free manner using a solid support. These sequence variants are constructed without the potential problems associated with passage of the viral constructions through cell cultures. Such a system will allow the amplification and isolation of nucleic acid sequences that are not well tolerated by bacteria in traditional cloning approaches.

Viruses are a unique class of infectious agents whose distinctive features are their simple organization and their mechanism of replication. Their hosts include a wide variety of plants and animals. A complete viral particle, or virion, may be regarded mainly as a block of genetic material (either DNA or RNA) capable of autonomous replication, surrounded by a protein coat and sometimes by an additional membranous envelope. The coat protects the virus from the environment and serves as a vehicle for transmission from one host cell to another.

### SUMMARY OF THE INVENTION

The present invention relates to a method for generating viral nucleic acids in a cell-free manner. In essence, the cell-free method entails the immobilization of a fragment of a double-stranded DNA sequence on a solid support and the assembly of the remaining fragments of the double-stranded DNA sequence onto the immobilized fragment. If the viral nucleic acid is derived from an RNA virus, the instant method further comprises the step of in vitro transcription of the assembled double-stranded DNA sequence to yield an RNA viral nucleic acid. The instant invention is particularly suitable for high throughput construction of viral nucleic acids. For example, the assembly of DNA fragments may be performed in a 96-, 384-, or 1536-well format.

One skilled in the art will appreciate that there are many ways to immobilize DNA fragments on a solid support and assemble fragments of a double-stranded DNA into its full length. In preferred embodiments of the instant invention, the assembly of DNA fragments is accomplished by first generating complementary single stranded ends for fragments of the double-stranded DNA, hybridizing one fragment with another fragment of the double-stranded DNA sequence, and ligating the two fragments of the double-stranded DNA sequence. This process is repeated until the full length DNA is assembled. The complementary single stranded ends are typically 1 to 15 nucleotides long.

In some embodiments of the instant invention, the viral nucleic acids may be divided into three fragments (the left arm, right arm and insert). One fragment, preferably the right arm corresponding to the 3′ portion of the viral nucleic acid, is immobilized on a solid support. A second fragment, preferably the insert, is assembled to the immobilized right arm. Preferably, the assembly method comprising the steps of generating complementary single stranded ends for fragments of the double-stranded DNA, hybridizing one fragment with another fragment of the double-stranded DNA sequence, and ligating the two fragments of the double-stranded DNA sequence. The third fragment, preferably the left arm corresponding to the 5′ portion of the viral nucleic acid, is assembled to the assembled right arm and the insert using similar methods. In particularly preferred embodiments, enzymes with 3′-5′ exonuclease activity may be used to generate complementary single stranded ends.

### BRIEF DESCRIPTION OF THE FIGURES

FIG. 1 illustrates the method for generating complementary single-stranded ends and assembling fragments of DNA sequences using T4 DNA polymerase (SEQ ID NOs: 1-12).

FIG. 2 illustrates a schematic diagram for constructing viral nucleic acids in a cell-free manner.

### DETAILED DESCRIPTION OF THE INVENTION

The present invention relates to a method for generating viral nucleic acids in a cell-free manner. In essence, the cell-free method entails the immobilization of a fragment of a double-stranded DNA sequence on a solid support and the assembly of the remaining fragments of the double-stranded DNA sequence onto the immobilized fragment. If the viral nucleic acid is derived from an RNA virus, the instant method further comprises the step of in vitro transcription of the assembled double-stranded DNA sequence to yield an RNA viral nucleic acid. The instant invention is particularly suitable for high throughput construction of viral nucleic acids. For example, the assembly of DNA fragments may be performed in a 96-, 384-, or 1536-well format.

I. Genetic Backbone and Components of Viral Nucleic Acids

A. Plant Viruses

One skilled in the art will appreciate that a variety of plant virus families may be used as the genetic backbone of the viral nucleic acids. These plant virus families may include Bromoviridae, Bunyaviridae, Comoviridae, Geminiviridae, Potyviridae, and Tombusviridae, among others. Within the plant virus families, various genera of viruses may be suitable for the instant invention, such as alfamovirus, ilarvirus, bromovirus, cucumovirus, tospovirus, carlavirus, caulimovirus, closterovirus, comovirus, nepovirus, dianthovirus, furovirus, hordeivirus, luteovirus, necrovirus, potexvirus, potyvirus, rymovirus, bymovirus, oryzavirus, sobemovirus, tobamovirus, tobravirus, carmovirus, tombusvirus, tymovirus, umbravirusa, and among others.

Within the genera of plant viruses, many species are particular preferred. They include alfalfa mosaic virus, tobacco streak virus, brome mosaic virus, broad bean mottle virus, cowpea chlorotic mottle virus, cucumber mosaic virus, tomato spotted wilt virus, carnation latent virus, caulflower mosaic virus, beet yellows virus, cowpea mosaic virus, tobacco ringspot virus, carnation ringspot virus, soil-borne wheat mosaic virus, tomato golden mosaic virus, cassava latent virus, barley stripe mosaic virus, barley yellow dwarf virus, tobacco necrosis virus, tobacco etch virus, potato virus X, potato virus Y, rice necrosis virus, ryegrass mosaic virus, barley yellow mosaic virus, rice ragged stunt virus, Southern bean mosaic virus, tobacco mosaic virus, ribgrass mosaic virus, cucumber green mottle mosaic virus watermelon strain, oat mosaic virus, tobacco rattle virus, carnation mottle virus, tomato bushy stunt virus, turnip yellow mosaic virus, carrot mottle virus, among others. In addition, RNA satellite viruses, such as tobacco necrosis satellite may also be employed.

A given plant virus may contain either DNA or RNA, which may be either single- or double-stranded. One example of plant viruses containing double-stranded DNA includes, but is not limited to, caulimoviruses such as Cauliflower mosaic virus (CaMV). Representative plant viruses which contain single-stranded DNA are cassava latent virus, bean golden mosaic virus (BGMV), and chloris striate mosaic virus. Rice dwarf virus and wound tumor virus are examples of double-stranded RNA plant viruses. Single-stranded RNA plant viruses include tobacco mosaic virus (TMV), turnip yellow mosaic virus (TYMV), rice necrosis virus (RNV) and brome mosaic virus (BMV). The single-stranded RNA viruses can be further divided into positive-stranded, negative-stranded, or ambisense viruses. The genomic RNA of a positive-stranded RNA virus is messenger sense, which makes the naked RNA infectious. Many plant viruses belong to the family of positive-stranded RNA viruses. They include, for example, TMV, BMV, and others. RNA plant viruses typically encode several common proteins, such as replicase/polymerase proteins essential for viral replication and mRNA synthesis, coat proteins providing protective shells for the extracellular passage, and other proteins required for the cell-to-cell movement, systemic infection and self-assembly of viruses. For general information concerning plant viruses, see Matthews, Plant Virology, 3^{rd} Ed., Academic Press, San Diego (1991).

Selected groups of suitable viruses are characterized below. However, the invention should not be construed as limited to using these particular viruses, but rather the method of the present invention is contemplated to include all plant viruses at a minimum.

### Tobamovirus Group

Tobacco Mosaic virus (TMV) is a member of the Tobamoviruses. The TMV virion is a tubular filament, and comprises coat protein sub-units arranged in a single right-handed helix with the single-stranded RNA intercalated between the turns of the helix. TMV infects tobacco as well as other plants. TMV is transmitted mechanically and may remain infective for a year or more in soil or dried leaf tissue.

The TMV virions may be inactivated by subjection to an environment with a pH of less than 3 or greater than 8, or by formaldehyde or iodine. Preparations of TMV may be obtained from plant tissues by (NH₄)₂SO₄ precipitation, followed by differential centrifugation.

Tobacco mosaic virus (TMV) is a positive-stranded ssRNA virus whose genome is 6395 nucleotides long and is capped at the 5′-end but not polyadenylated. The genomic RNA can serve as mRNA for protein of a molecular weight of about 130,000 (130K) and another produced by read-through of molecular weight about 180,000 (180K). However, it cannot function as a messenger for the synthesis of coat protein. Other genes are expressed during infection by the formation of monocistronic, 3′-coterminal subgenomic mRNAs, including one (LMC) encoding the 17.5K coat protein and another (I₂) encoding a 30K protein. The 30K protein has been detected in infected protoplasts as described in Miller, J., Virology 132:71 (1984), and it is involved in the cell-to-cell transport of the virus in an infected plant as described by Deom et al., Science 237:389 (1987). The functions of the two large proteins are unknown, however, they are thought to function in RNA replication and transcription.

Several double-stranded RNA molecules, including double-stranded RNAs corresponding to the genomic, I₂ and LMC RNAs, have been detected in plant tissues infected with TMV. These RNA molecules are presumably intermediates in genome replication and/or mRNA synthesis processes which appear to occur by different mechanisms.

TMV assembly apparently occurs in plant cell cytoplasm, although it has been suggested that some TMV assembly may occur in chloroplasts since transcripts of ctDNA have been detected in purified TMV virions. Initiation of TMV assembly occurs by interaction between ring-shaped aggregates (“discs”) of coat protein (each disc consisting of two layers of 17 subunits) and a unique internal nucleation site in the RNA; a hairpin region about 900 nucleotides from the 3′-end in the common strain of TMV. Any RNA, including subgenomic RNAs containing this site, may be packaged into virions. The discs apparently assume a helical form on interaction with the RNA, and assembly (elongation) then proceeds in both directions (but much more rapidly in the 3′- to 5′-direction from the nucleation site).

Another member of the Tobamoviruses, the Cucumber Green Mottle Mosaic virus watermelon strain (CGMMV-W) is related to the cucumber virus. Nozu et al., Virology 45:577 (1971). The coat protein of CGMMV-W interacts with RNA of both TMV and CGMMV to assemble viral particles in vitro. Kurisu et al., Virology 70:214 (1976).

Several strains of the tobamovirus group are divided into two subgroups, on the basis of the location of the origin of assembly. Subgroup I, which includes the vulgare, OM, and tomato strain, has an origin of assembly about 800-1000 nucleotides from the 3′-end of the RNA genome, and outside the coat protein cistron. Lebeurier et al., Proc. Natl. Acad. Sci. USA 74:149 (1977); and Fukuda et al., Virology 101:493 (1980). Subgroup II, which includes CGMMV-W and cowpea strain (Cc) has an origin of assembly about 300-500 nucleotides from the 3′-end of the RNA genome and within the coat protein cistron. The coat protein cistron of CGMMV-W is located at nucleotides 176-661 from the 3′-end. The 3′ noncoding region is 175 nucleotides long. The origin of assembly is positioned within the coat protein cistron. Meshi et al., Virology 127:54 (1983).

### Brome Mosaic Virus Group

Brome Mosaic virus (BMV) is a member of a group of tripartite, single-stranded, RNA-containing plant viruses commonly referred to as the bromoviruses. Each member of the bromoviruses infects a narrow range of plants. Mechanical transmission of bromoviruses occurs readily, and some members are transmitted by beetles. In addition to BMV, other bromoviruses include broad bean mottle virus and cowpea chlorotic mottle virus.

Typically, a bromovirus virion is icosahedral, with a diameter of about 26 μm, containing a single species of coat protein. The bromovirus genome has three molecules of linear, positive-sense, single-stranded RNA, and the coat protein mRNA is also encapsidated. The RNAs each have a capped 5′-end, and a tRNA-like structure (which accepts tyrosine) at the 3′-end. Virus assembly occurs in the cytoplasm. The complete nucleotide sequence of BMV has been identified and characterized as described by Ahlquist et al., J Mol. Biol. 153:23 (1981).

### Rice Necrosis Virus

Rice Necrosis virus is a member of the Potato Virus Y Group or Potyviruses. The Rice Necrosis virion is a flexuous filament comprising one type of coat protein (molecular weight about 32,000 to about 36,000) and one molecule of linear positive-sense single-stranded RNA. The Rice Necrosis virus is transmitted by Polymyxa oraminis (a eukaryotic intracellular parasite found in plants, algae and fungi).

### Geminiviruses

Geminiviruses are a group of small, single-stranded DNA-containing plant viruses with virions of unique morphology. Each virion consists of a pair of isometric particles (incomplete icosahedral), composed of a single type of protein (with a molecular weight of about 2.7-3.4×10⁴). Each geminivirus virion contains one molecule of circular, positive-sense, single-stranded DNA. In some geminiviruses (i.e., Cassava latent virus and bean golden mosaic virus) the genome appears to be bipartite, containing two single-stranded DNA molecules.

### Potyviruses

Potyviruses are a group of plant viruses which produce polyprotein. A particularly preferred potyvirus is tobacco etch virus (TEV). TEV is a well characterized potyvirus and contains a positive-strand RNA genome of 9.5 kilobases encoding for a single, large polyprotein that is processed by three virus-specific proteinases. The nuclear inclusion protein “a” proteinase is involved in the maturation of several replication-associated proteins and capsid protein. The helper component-proteinase (HC-Pro) and 35-kDa proteinase both catalyze cleavage only at their respective C-termini. The proteolytic domain in each of these proteins is located near the C-terminus. The 35-kDa proteinase and HC-Pro derive from the N-terminal region of the TEV polyprotein.

The selection of genetic backbone for the viral nucleic acids of the instant invention may depend on the plant host used. The plant host may be a monocotyledonous or dicotyledonous plant, plant tissue, or plant cell. Typically, plants of commercial interest, such as food crops, seed crops, oil crops, ornamental crops and forestry crops are preferred. For example, wheat, rice, corn, potato, barley, tobacco, soybean canola, maize, oilseed rape, lilies, grasses, orchids, irises, onions, palms, tomato, the legumes, or Arabidopsis, can be used as a plant host. can be used as a host plant. Host plants may also include those readily infected by an infectious virus, such as Nicotiana, preferably, Nicotiana benthamina, or Nicotiana clevelandii.

One feature of the present invention is the use of plant viral nucleic acids which comprise one or more non-native nucleic acid sequences capable of being transcribed in a plant host. These nucleic acid sequences may be native nucleic acid sequences that occur in a host plant. Preferably, these nucleic acid sequences are non-native nucleic acid sequences that do not normally occur in a host plant. For example, the plant viral vectors may contain sequences from more than one virus, including viruses from more than one taxonomic group. The plant viral nucleic acids may also contain sequences from non-viral sources, such as foreign genes, regulatory sequences, fragments thereof from bacteria, fungi, plants, animals or other sources. These foreign sequences may encode commercially useful proteins, polypeptides, or fusion products thereof, such as enzymes, antibodies, hormones, pharmaceuticals, vaccines, pigments, and the like. Or they may be sequences that regulate the expression of foreign genes, package viral nucleic acids, and facilitate systemic infection in the host, etc.

In some embodiments of the instant invention, the plant viral vectors may comprise one or more additional native or non-native subgenomic promoters which are capable of transcribing or expressing adjacent nucleic acid sequences in the plant host. These non-native subgenomic promoters are inserted into the plant viral nucleic acid without destroying the biological function of the plant viral nucleic acid using known methods in the art. For example, the CaMV promoter can be used when plant cells are to be transfected. The subgenomic promoters are capable of functioning in the specific host plant. For example, if the host is tobacco, TMV, tomato mosaic virus, or other viruses containing subgenomic promoter may be utilized. The inserted subgenomic promoters should be compatible with the TMV nucleic acid and capable of directing transcription or expression of adjacent nucleic acid sequences in tobacco. It is specifically contemplated that two or more heterologous non-native subgenomic promoters may be used. The non-native nucleic acid sequences may be transcribed or expressed in the host plant under the control of the subgenomic promoter to produce the products of the nucleic acids of interest.

In some embodiments of the instant invention, the recombinant plant viral nucleic acids may be further modified by conventional techniques to delete all or part of the native coat protein coding sequence or put the native coat protein coding sequence under the control of a non-native plant viral subgenomic promoter. If it is deleted or otherwise inactivated, a non-native coat protein coding sequence is inserted under control of one of the non-native subgenomic promoters, or optionally under control of the native coat protein gene subgenomic promoter. Thus, the recombinant plant viral nucleic acid contains a coat protein coding sequence, which may be native or a nonnative coat protein coding sequence, under control of one of the native or non-native subgenomic promoters. The native or non-native coat protein gene may be utilized in the recombinant plant viral nucleic acid. The non-native coat protein, as is the case for the native coat protein, may be capable of encapsidating the recombinant plant viral nucleic acid and providing for systemic spread of the recombinant plant viral nucleic acid in the host plant.

In some embodiments of the instant invention, recombinant plant viral vectors are constructed to express a fusion between a plant viral coat protein and the foreign genes or polypeptides of interest. Such a recombinant plant virus provides for high level expression of a nucleic acid of interest. The location(s) where the viral coat protein is joined to the amino acid product of the nucleic acid of interest may be referred to as the fusion joint. A given product of such a construct may have one or more fusion joints. The fusion joint may be located at the carboxyl terminus of the viral coat protein or the fusion joint may be located at the amino terminus of the coat protein portion of the construct. In instances where the nucleic acid of interest is located internal with respect to the 5′ and 3′ residues of the nucleic acid sequence encoding for the viral coat protein, there are two fusion joints. That is, the nucleic acid of interest may be located 5′, 3′, upstream, downstream or within the coat protein. In some embodiments of such recombinant plant viruses, a “leaky” start or stop codon may occur at a fusion joint which sometimes does not result in translational termination.

In some embodiments of the instant invention, nucleic sequences encoding reporter protein(s) or antibiotic/herbicide resistance gene(s) may be constructed as carrier protein(s) for the polypeptides of interest, which may facilitate the detection of polypeptides of interest. For example, green fluorescent protein (GFP) may be simultaneously expressed with polypeptides of interest. In another example, a reporter gene, β-glucuronidase (GUS) may be utilized. In another example, a drug resistance marker, such as a gene whose expression results in kanamycin resistance, may be used.

In some embodiment of the instant invention, the RNA is capped using conventional techniques, if the capped RNA is the infective agent. In addition, the capped RNA can be packaged in vitro with added coat protein from a plant virus to make assembled virions. These assembled virions can then be used to inoculate plants or plant tissues. Alternatively, an uncapped RNA may also be employed in some embodiments of the present invention. Contrary to the practiced art in scientific literature and in issued patent (Ahlquist et al., U.S. Pat. No. 5,466,788), uncapped transcripts for virus expression vectors are infective on both plants and in plant cells. Capping is not a prerequisite for establishing an infection of a virus expression vector in plants, although capping increases the efficiency of infection. In addition, nucleotides may be added between the transcription start site of the promoter and the start of the cDNA of a viral nucleic acid to construct an infectious viral vector. One or more nucleotides may be added. In some embodiments of the present invention, the inserted nucleotide sequence may contain a G at the 5′-end. Alternatively, the inserted nucleotide sequence may be GNN, GTN, or their multiples, (GNN)ₓ or (GTN)ₓ.

In some embodiments of the instant invention, more than one nucleic acid is prepared for a multipartite viral vector construct. In this case, each nucleic acid may require its own origin of assembly. Each nucleic acid could be prepared to contain a subgenomic promoter and a non-native nucleic acid. Alternatively, the insertion of a non-native nucleic acid into the nucleic acid of a monopartite virus may result in the creation of two nucleic acids (i.e., the nucleic acid necessary for the creation of a bipartite viral vector). This would be advantageous when it is desirable to keep the replication and transcription or expression of the nucleic acid of interest separate from the replication and translation of some of the coding sequences of the native nucleic acid.

B. Animal Viruses

One skilled in the art will appreciate that the viral nucleic acids may also be derived from a variety of animal viruses, such as an alphavirus, rhinovirus, poliovirus, polyomavirus, simian virus 40, adenovirus, baculoviruses, and nodaviruses among others. Selected groups of suitable viruses are characterized below. However, the invention should not be construed as limited to using these particular viruses, but rather the method of the present invention is contemplated to include all animal viruses at a minimum.

### Alphaviruses

The alphaviruses are a genus of the viruses of the family Togaviridae. Almost all of the members of this genus are transmitted by mosquitoes, and may cause diseases in man or animals. Some of the alphaviruses are grouped into three serologicallly defined complexes. The complex-specific antigen is associated with the E1 protein of the virus, and the species-specific antigen is associated with the E2 protein of the virus.

The Semliki Forest virus complex includes Bebaru virus, Chikungunya Fever virus, Getah virus, Mayaro Fever virus, O'nyongnyong Fever virus, Ross River virus, Sagiyama virus, Semliki Forest virus and Una virus. The Venezuelan Equine Encephalomyelitis virus complex includes Cabassou virus, Everglades virus, Mucambo virus, Pixuna virus and Venezuelan Equine Encephalomyelitis virus. The Western Equine Encephalomyelitis virus complex includes Aura virus, Fort Morgan virus, Highlands J virus, Kyzylagach virus, Sindbis virus, Western Equine Encephalomyelitis virus and Whataroa virus.

The alphaviruses contain an icosahedral nucleocapsid consisting of 180 copies of a single species of capsid protein complexed with a plus-stranded mRNA. The alphaviruses mature when preassembled nucleocapsid is surrounded by a lipid envelope containing two virus-encoded integral membrane glycoproteins, called E1 and E2. The envelope is acquired when the capsid, assembled in the cytoplasm, buds through the plasma membrane. The envelope consists of a lipid bilayer derived from the host cell.

The mRNA encodes a glycoprotein which is cotranslationally cleaved into nonstructural proteins and structural proteins. The 3′ one-third of the RNA genome consists of a 26S mRNA which encodes for the capsid protein and the E3, E2, K6 and E1 glycoproteins. The capsid is cotranslationally cleaved from the E3 protein. It is hypothesized that the amino acid triad of His, Asp and Ser at the COOH terminus of the capsid protein comprises a serine protease responsible for cleavage. Hahn et al., Proc. Natl. Acad. Sci. USA 82:4648 (1985). Cotranslational cleavage also occurs between E2 and K proteins. Thus, two proteins PE2 which consists of E3 and E2 prior to cleavage and an E1 protein comprising K6 and E1 are formed. These proteins are cotranslationally inserted into the endoplasmic reticulum of the host cell, glycosylated and transported via the Golgi apparatus to the plasma membrane where they can be used for budding. At the point of virion maturation the E3 and E2 proteins are separated. The E1 and E2 proteins are incorporated into the lipid envelope.

It has been suggested that the basic amino-terminal half of the capsid protein stabilizes the interaction of capsid with genomic RNA or interacts with genomic RNA to initiate a encapsidation, Strauss et al, in the Togaviridae and Flaviviridaei, Ed. S. Schlesinger & M. Schlesinger, Plenum Press, New York, pp. 35-90 (1980). These suggestions imply that the origin of assembly is located either on the unencapsidated genomic RNA or at the amino-terminus of the capsid protein. It has been suggested that E3 and K6 function as signal sequences for the insertion of PE2 and E1, respectively, into the endoplasmic reticulum.

Work with temperature sensitive mutants of alphaviruses has shown that failure of cleavage of the structural proteins results in failure to form mature virions. Lindquist et al., Virology 151:10 (1986) characterized a temperature sensitive mutant of Sindbis virus, tₛ 20. Temperature sensitivity results from an A-U change at nucleotide 9502. The t_{S} lesion present cleavage of PE2 to E2 and E3 and the final maturation of progeny virions at the nonpermissive temperature. Hahn et al., supra, reported three temperature sensitive mutations in the capsid protein which prevents cleavage of the precursor polyprotein at the nonpermissive temperature. The failure of cleavage resulted in no capsid formation and very little envelope protein.

Defective interfering RNAs (DI particles) of Sindbis virus are helper-dependent deletion mutants which interfere specifically with the replication of the homologous standard virus. Perrault, J., Microbiol. Immunol. 93:151 (1981). DI particles have been found to be functional vectors for introducing at least one foreign gene into cells. Levis, R., Proc. Natl. Acad. Sci. USA 84:4811 (1987).

It has been found that it is possible to replace at least 1689 internal nucleotides of a DI genome with a foreign sequence and obtain RNA that will replicate and be encapsidated. Deletions of the DI genome do not destroy biological activity. The disadvantages of the system are that DI particles undergo apparently random rearrangements of the internal RNA sequence and size alterations. Monroe et al., J. Virology 49:865 (1984). Expression of a gene inserted into the internal sequence is not as high as expected. Levis et al., supra, found that replication of the inserted gene was excellent but translation was low. This could be the result of competition with whole virus particles for translation sites and/or also from disruption of the gene due to rearrangement through several passages.

Two species of mRNA are present in alphavirus-infected cells: A 42S mRNA region, which is packaged into nature virions and functions as the message for the nonstructural proteins, and a 26S mRNA, which encodes the structural polypeptides. the 26S mRNA is homologous to the 3′ third of the 42S mRNA. It is translated into a 130K polyprotein that is cotranslationally cleaved and processed into the capsid protein and two glycosylated membrane proteins, E1 and E2.

The 26S mRNA of Eastern Equine Encephalomyelitis (EEE) strain 82V-2137 was cloned and analyzed by Chang et al., J. Gen. Virol 68:2129 (1987). The 26S mRNA region encodes the capsid proteins, E3, E2, 6K and E1. The amino terminal end of the capsid protein is thought to either stabilize the interaction of capsid with mRNA or to interact with genomic RNA to initiate encapsidation.

Uncleaved E3 and E2 proteins called PE2 is inserted into the host endoplasmic reticulum during protein synthesis. The PE2 is thought to have a region common to at least five alphaviruses which interacts with the viral nucleocapsid during morphogenesis.

The 6K protein is thought to function as a signal sequence involved in translocation of the E1 protein through the membrane. The E1 protein is thought to mediate virus fusion and anchoring of the E1 protein to the virus envelope.

### Rhinoviruses

The rhinoviruses are a genus of viruses of the family Picornaviridae. The rhinoviruses are acid-labile, and are therefore rapidly inactivated at pH values of less than about 6. The rhinoviruses commonly infect the upper respiratory tract of mammals.

Human rhinoviruses are the major causal agents of the common cold, and many serotypes are known. Rhinoviruses may be propagated in various human cell cultures, and have an optimum growth temperature of about 33° C. Most strains of rhinoviruses are stable at or below room temperature and can withstand freezing. Rhinoviruses can be inactivated by citric acid, tincture of iodine or phenol/alcohol mixtures.

The complete nucleotide sequence of human rhinovirus 2 (HRV2) has been sequenced. The genome consists of 7102 nucleotides with a long open reading frame of 6450 nucleotides which is initiated 611 nucleotides from the 5′-end and stops 42 nucleotides from the poly(A) tract. Three capsid proteins and their cleavage cites have been identified.

Rhinovirus RNA is single-stranded and positive-sense. The RNA is not capped, but is joined at the 5′-end to a small virus-encoded protein, virion-protein genome-linked (VPg). Translation is presumed to result in a single polyprotein which is broken by proteolytic cleavage to yield individual virus proteins. An icosahedral viral capsid contains 60 copies each of 4 virus proteins VP1, VP2, VP3 and VP4 and surrounds the RNA genome. Medappa, K., Virology 44:259 (1971).

Analysis of the 610 nucleotides preceding the long open reading frame shows several short open reading frames. However, no function can be assigned to the translated proteins since only two sequences show homology throughout HRV2, HRV14 and the 3 sterotypes of poliovirus. These two sequences may be critical in the life cycle of the virus. They are a stretch of 16 bases beginning at 436 in HRV2 and a stretch of 23 bases beginning at 531 in HRV2. Cutting or removing these sequences from the remainder of the sequence for non-structural proteins could have an unpredictable effect upon efforts to assemble a mature virion.

The capsid proteins of HRV2: VP4, VP2, VP3 and VP 1 begin at nucleotide 611, 818, 1601 and 2311, respectively. The cleavage point between VP1 and P2A is thought to be around nucleotide 3255. Skern et al., Nucleic Acids Research 13:2111 (1985).

Human rhinovirus type 89 (HRV89) is very similar to HRV2. It contains a genome of 7152 nucleotides with a single large open reading frame of 2164 condons. Translation begins at nucleotide 619 and ends 42 nucleotides before the poly(A) tract. The capsid structural proteins, VP4, VP2, VP3 and VP1 are the first to be translated. Translation of VP4 begins at 619. Cleavage cites occur at:

| | | |
|---|---|---|
| VP4/VP2 | 825 | determined |
| VP2/VP3 | 1627 | determined |
| VP3/VP1 | 2340 | determined |
| VP1/P2-A | 3235 | presumptive |

Duechler et al., Proc. Natl. Acad. Sci. USA 84:2605 (1987).

### Polioviruses

Polioviruses are the causal agents of poliomyelitis in man, and are one of three groups of enteroviruses. Enteroviruses are a genus of the family Picornaviridae (also the family of rhinoviruses). Most enteroviruses replicate primarily in the mammalian gastrointestinal tract, although other tissues may subsequently become infected. Many enteroviruses can be propagated in primarily cultures of human or monkey kidney cells and in some cell lines (e.g. HeLa, Vero, WI-e8). Inactivation of the enteroviruses may be accomplished with heat (about 50° C.), formaldehyde (3%), hydrochloric acid (0.1N) or chlorine (ca. 0.3-0.5 ppm free residual Cl₂).

The complete nucleotide sequence of poliovirus PV2 (Sab) and PV3 (Sab) have been determined. They are 7439 and 7434 nucleotide in length, respectively. There is a single long open reading frame which begins more than 700 nucleotides from the 5′-end. Poliovirus translation produces a single polyprotein which is cleaved by proteolytic processing. Kitamura et al., Nature 291:547 (1981).

It is speculated that these homologous sequences in the untranslated regions play an essential role in viral replication such as:

1. viral-specific RNA synthesis;

2. viral-specific protein synthesis; and

3. packaging

Toyoda, H. et al., J Mol. Biol. 174:561 (1984).

The structures of the serotypes of poliovirus have a high degree of sequence homology. Their coding sequences code for the same proteins in the same order. Therefore, genes for structural proteins are similarly located. In PV1, PV2 and PV3, the polyprotein begins translation near the 750 nucleotide. The four structural proteins VP4, VP2, VP3 and VP1 begin at about 745, 960, 1790 and 2495, respectively, with VPI ending at about 3410. They are separated in vivo by proteolytic cleavage, rather than by stop/start codons.

### Simian Virus 40

Simian virus 40 (SV40) is a virus of the genus Polyomavirus, and was originally isolated from the kidney cells of the rhesus monkey. The virus is commonly found, in its latent form, in such cells. Simian virus 40 is usually non-pathogenic in its natural host.

Simian virus 40 virions are made by the assembly of three structural proteins, VP1, VP2 and VP3. Girard et al, Biochem. Biophys. Res. Commun. 40:97 (1970); Prives et al., Proc. Natl. Acad. Sci. USA 71:302 (1974); and Jacobson et al., Proc. Natl. Acad. Sci. USA 73:2742-2746 (1976). The three corresponding viral genes are organized in a partially overlapping manner. They constitute the late genes portion of the genome. Tooze, J., Molecular Biology of Tumor Viruses Appendix A The SV40 Nucleotide Sequence, 2nd Ed. Part 2, pp. 799-829 (1980), Cold Spring Harbor Laboratory, Cold Spring Harbor, N.Y. Capsid proteins VP2 and VP3 are encoded by nucleotides 545 to 1601 and 899 to 1601, respectively, and both are read in the same frame. VP3 is therefore a subset of VP2. Capsid protein VP1 is encoded by nucleotides 1488-2574. The end of the VP2-VP3 open reading frame therefore overlaps the VP1 by 113 nucleotides but is read in an alternative frame. Tooze, J., supra. Wychowski et al., J. Virology 61:3862 (1987).

### Adenoviruses

Adenovirus type 2 is a member of the adenovirus family or adenovirus. This family of viruses are non-enveloped, icosahedral, linear, double-stranded DNA-containing viruses which infect mammals or birds.

The adenovirus virion consists of an icosahedral capsid enclosing a core in which the DNA genome is closely associated with a basic (arginine-rich) viral polypeptide VII. The capsid is composed of 252 capsomeres: 240 hexons (capsomers each surrounded by 6 other capsomers) and 12 pentons (one at each vertex, each surrounded by 5 ‘peripentonal’ hexons). Each penton consists of a penton base (composed of viral polypeptide III) associated with one (in mammalian adenoviruses) or two (in most avian adenoviruses) glycoprotein fibres (viral polypeptide IV). The fibres can act as haemagglutinins and are the sites of attachment of the virion to a host cell-surface receptor. The hexons each consist of three molecules of viral polypeptide II; they make up the bulk of the icosahedron. Various other minor viral polypeptides occur in the virion.

The adenovirus dsDNA genome is covalently linked at the 5′-end of each strand to a hydrophobic ‘terminal protein’, TP (molecular weight about 55,000 Da); the DNA has an inverted terminal repeat of different length in different adenoviruses. In most adenoviruses examined, the 5′-terminal residue is dCMP.

During its replication cycle, the virion attaches via its fibres to a specific cell-surface receptor, and enters the cell by endocytosis or by direct penetration of the plasma membrane. Most of the capsid proteins are removed in the cytoplasm. The virion core enters the nucleus, where the uncoating is completed to release viral DNA almost free of virion polypeptides. Virus gene expression then begins. The viral dsDNA contains genetic information on both strands. Early genes (regions E1a, E1b, E2a, E3, E4) are expressed before the onset of viral DNA replication. Late genes (regions L1, L2, L3, L4 and L5) are expressed only after the initiation of DNA synthesis. Intermediate genes (regions E2b and Iva₂) are expressed in the presence or absence of DNA synthesis. Region E1a encodes proteins involved in the regulation of expression of other early genes, and is also involved in transformation. The RNA transcripts are capped (with m⁷G⁵ppp⁵N) and polyadenylated in the nucleus before being transferred to the cytoplasm for translation.

Viral DNA replication requires the terminal protein, TP, as well as virus-encoded DNA polymerase and other viral and host proteins. TP is synthesized as an 80K precursor, pTP, which binds covalently to nascent replicating DNA strands. pTP is cleaved to the mature 55K TP late in virion assembly; possibly at this stage, pTP reacts with a dCTP molecule and becomes covalently bound to a dCMP residue, the 3′ OH of which is believed to act as a primer for the initiation of DNA synthesis. Late gene expression, resulting in the synthesis of viral structural proteins, is accompanied by the cessation of cellular protein synthesis, and virus assembly may result in the production of up to 10⁵ virions per cell.

### Baculoviruses

Baculoviruses are a group of viruses of the family Baculoviridae that have been used to express foreign proteins in insect cells. Baculovirus vectors derived from the Autographa californica multiple nuclear polyhedrosis virus (AcMNPV) have a host range that is limited to the order lepidoptera. NPVs contain a circular, double-stranded, supercoiled DNA genome that is packaged into a rod shaped virion. NPVs replicate in the nuclei and form polyhedral occlusion bodies. Baculovirus expression vectors derived from the Autographa californica nuclear polyhedrosis virus have been used for many years to overexpress recombinant proteins in insect-derived host cells. Recombinant baculoviruses can serve as gene-transfer vehicles for transient expression of recombinant proteins in a wide range of mammalian cell types. By inclusion of a dominant selectable marker in the viral vector, cell lines can be derived that stably express recombinant genes. Condreay et al (Proc. Natl., Acad., Sci. USA, 96: 127-132 (1999)) constructed a recombinant baculovirus containing two expression cassettes controlled by constitutive mammalian promoters: the cytomegalovirus immediate early promoter/enhancer directing expression of green fluorescent protein and the simian virus 40 early promoter controlling neomycin phosphotransferase II. Using this virus, efficient gene delivery and expression was observed and measured in numerous cell types of human, primate, and rodent origin.

### Nodaviruses

Nodaviruses are a group of small (+) strand RNA viruses that infect both invertebrates and vertebrates. Flock house virus (FLV), a member of the Nodaviridae family, can infect insect, plant, and mammalian cells. Infectious transcripts can be produced in vitro and in vivo. De novo synthesis of FLV virions has been demonstrated in S. cerevisiae (Price et al., Proc. Natl., Acad., Sci. USA 93: 9465-9570, (1996)).

Those skilled in the art will understand that these embodiments are representative only of many constructs suitable for the instant invention. All such constructs are contemplated and intended to be within the scope of the present invention. The invention is not intended to be limited to any particular viral constructs but specifically contemplates using all operable constructs. A person skilled in the art will be able to construct the plant viral vectors based on molecular biology techniques well known in the art. Suitable techniques have been described in Sambrook et al. (2nd ed.), Cold Spring Harbor Laboratory, Cold Spring Harbor (1989); Methods in Enzymol. (Vols. 68, 100, 101, 118, and 152-155) (1979, 1983, 1986 and 1987); and DNA Cloning, D. M. Clover, Ed., IRL Press, Oxford (1985); Walkey, Applied Plant Virology, Chapman & Hall (1991); Matthews, Plant Virology, 3^{rd} Ed., Academic Press, San Diego (1991); Turpen et al., J. of Virological Methods, 42:227-240 (1993); U.S. Pat. Nos. 4,885,248, 5,173,410, 5,316,931, 5,466,788, 5,491,076, 5,500,360, 5,589,367, 5,602,242, 5,627,060, 5,811,653, 5,866,785, 5,889,190, and 5,589,367, and U.S. patent application Ser. No. 08/324,003. Nucleic acid manipulations and enzyme treatments are carried out in accordance with manufacturers' recommended procedures in making such constructs.

II. Generating Libraries of Nucleic Acid Sequence Variants

One or more template sequences may be used to generate libraries of nucleic acid sequence variants via in vitro mutagenesis, recombination or a combination thereof. In some embodiments of the invention, the template sequences may be derived from elements of plant viruses, such as the coat protein, movement protein, promoter sequences, internal initiation sites, packaging signals, 5′and 3′ NTRs, ribosomal sequences, among others. In preferred embodiments, elements of the open reading frame (ORF) of RNA plant viruses is the starting point for sequence variation. Functions within the ORF include the movement protein (MP), the virus origin of virion assembly, the subgenomic promoter used for coat protein synthesis, among others. The entire plant virus genomes may also be subjected to randomization so to improve plant viral nucleic acid performance.

In other embodiments of the invention, genes, regulatory sequences, or fragments thereof from prokaryotic and eukaryotic sources, such as bacteria, fungi, plants, animals, animal viruses, among others may serve as template sequences for generating sequence variants. For example, sequences regulating the transcription and translation of commercially useful proteins, polypeptides, or fusion products thereof, such as enzymes, antibodies, hormones, pharmaceuticals, vaccines, pigments, antimicrobial polypeptides, and the like may be used as templates to generate libraries of sequence variants.

One skilled in the art will appreciate that there are many ways to generate sequence variants. A population of nucleic acid sequence variants may be found in nature. For example, a genomic library, cDNA library, a pool of RNAs derived from bacteria, fungi, plants, or animals including humans, may be constructed. A more detailed discussion of generating such library is presented in a co-pending and co-owned U.S. patent application Ser. No. 09/359,300, incorporated herein by reference. In some instances, natural sequence variations may consist of different alleles of the same gene or the same gene from different related species. Alternatively, they may be related nucleic acid sequences found within one species, for example, the immunoglobulin genes. In addition, the natural variations in plant and animal viral populations may also be the templates for generating sequence libraries.

In preferred embodiments, the sequence variants may be generated using in vitro methods, including, but not limited to, chemical treatment, oligonucleotide mediated mutagenesis, PCR mutagenesis, DNA shuffling, random-priming recombination, restriction enzyme fragment induced template switching, staggered extension process, and other in vitro recombination methods. The sequence populations may be random or selectively varied.

The nucleic acid sequence can be altered by chemical mutagenesis. Chemical mutagens include, for example, sodium bisulfite, nitrous acid, hydroxylamine, hydrazine or formic acid. Other agents which are analogues of nucleotide precursors include nitrosoguanidine, 5-bromouracil, 2-aminopurine, or acridine. In some embodiemnts, these agents may be added to the PCR reaction in place of the nucleotide precursor thereby mutating the sequence. Intercalating agents such as proflavine, acriflavine, quinacrine and the like can also be used. Random mutagenesis of the nucleic acid sequence can also be achieved by irradiation with X-rays or ultraviolet light.

In oligonucleotide-directed mutagenesis, a short synthetically mutagenized oligonucleotide incorporating the desired base changes is hybridized to the sequences to be altered (Sambrook, et al., Molecular Cloning: A Laboratory Manual, 2^{nd} Ed. Cold Spring Harbor Laboratory Press, Cold Spring Harbor, N.Y., 1989 and Cleland et al., Protein Engineering: Principles and Practice, Wiley-Liss (1996)). The mismatched primer is then extended by polymerase, thereby generating the varied sequence. Individually varied sequences may be mixed together and expressed to select the desired function. This approach is particularly useful in generating sequence variations that are close to each other.

Error-prone PCR may be employed to create libraries of point mutations (Eckert et al., PCR Methods App. 1:17-24(1991); Caldwell et al., PCR Methods App. 2:28-33 (1992), Gramm et al., Proc. Natl. Acad. Sci. USA 89:3576-3580 (1992); and Cadwell et al., PCR Methods App. 3:S136-40 (1994); You et al. Protein Eng. 9:77-83 (1994)). This method uses a low fidelity replication to introduce random point mutations at each round of amplification. Repeated cycles of error-prone PCR may lead to accumulation of point mutations. Error prone PCR can be used to mutagenize a mixture of template sequences blindly without knowing their nucleotide composition. Error-prone PCR is particularly suited when regions of mutagenesis are small, typically less than 1,000 bp.

Combinatorial cassette mutagenesis (Black et al., Proc. Natl. Acad. Sci. USA 93:3525-3529 (1996) and recursive ensemble mutagenesis (Delagrave et al., Biotechnology 11:1548-1552 (1993) and Arkin et al., Proc. Natl. Acad. Sci. USA 89:7811-7815 (1992)) may also be used to produce sequence variances. In cassette mutagenesis, a sequence block of a single template is typically replaced by a randomized or partially randomized sequence. Therefore, sequence variants are typically determined by the size of the sequence block and the number of random sequences. The randomized sequences may be derived from synthetically mutagenized oligonucleotides. Typically, the nucleotide compositions of the template sequences are known.

Nucleic acid shuffling is a method for in vitro homologous recombination of pools of nucleic acid sequence variants (U.S. Pat. Nos. 5,830,721, 5,811,238, 5,830,721, 5,605,793, 5,834,252, and 5,837,458). This procedure involves random fragmentation of mixtures of related nucleic acid sequences followed by reassembly to yield a population of nucleic acid sequence variants.

Random-priming recombination (RPR) is another simple and efficient method for in vitro recombination of nucleic acid sequences is random-priming recombination (RPR) (Shao et al., Nucleic Acids Res. 26:681-683 (1998)). In this method, random sequence primers are used to generate a large number of short DNA fragments complementary to different sections of the template sequences. Due to base misincorporation and mispriming, these short DNA fragments also contain a low level of point mutations. The short DNA fragments may prime one another based on homology, and be recombined and reassembled by repeated cycles of denaturation, annealing and further enzyme-catalyzed DNA polymerization to produce a library of full-length sequences.

Restriction enzyme fragment induced template switching (REFITS) is a technically simple means of in vitro recombination between homologous DNA sequences. One of the technical challenges in DNA shuffling is reproducible generation of fragments of the appropriate size by Dnase. The DnaseI reaction is very sensitive to variations in template and enzyme concentrations. REFITS provides a different approach to generating fragments that is much easier to reproduce. It is a method to increase the rate of molecular evolution via in vitro homologous recombination of pools of mutant genes by fragmentation of the DNA with restriction enzymes and reassembly of fragments by PCR. The technique may be used to recombine homologous genes from related organisms, or to reassort random mutations, such as those generated by error-prone PCR.

The target DNA may be split into aliquots, and each aliquot is digested with a different restriction enzyme, or groups of restriction enzymes that cut the target DNA several times. Preferably, the restriction enzymes used in REFITS have four-base recognition site. In preferred embodiments, restriction enzymes are chosen to avoid large uncut fragments to improve the resolution of the recombination and help make sure that no large region remains unshuffled. The resolution of the recombination is determined by how close two mutations can be and still be separated and recombined at a detectable level. The resolution is also increased by using more enzymes to generate more pools of fragments. Since each separate digestion is done to completion, no careful timing of digestion is required, unlike Dnase I partial digestion. Some partial digestion products may also be tolerated by the REFITS procedure.

Staggered extension process (StEP) is another simple and efficient method for in vitro recombination of polynucleotide sequences to generate libraries of sequence variants (Zhao et al., Nat. Biotechnol. 16:258-261 (1998)). Rather than reassembling recombined sequences from a pool of fragmented template sequences, StEP prepares full-length recombined genes in the presence of the templates. Essentially, StEP consists of priming the template sequences followed by repeated cycles of denaturation and extremely abbreviated annealing/polymerase-catalyzed extension. This limited polymerase extension time is used to generate less-than-full-length fragments. In each cycle the growing fragments anneal to different templates based on sequence complenmentarity and extend further to create “recombination cassettes.” This is repeated until full-length sequences form. Due to template switching, most of the DNA contain sequence information from different parental sequences. The speed of the thermal cycle may be adjusted to avoid the polymerase adding too many bases at each cycle. Adding too many bases at each cycle may limit the amount of possible template switches and so limiting the amount of recombination and resolution between template switches. StEP may be performed using flanking universal primers to avoid bias introduced from the starting primers. A detailed discussion of methods for generating libraries of nucleic acid sequence variants and expressing such in plant hosts is presented in two co-pending and co-owned U.S. patent application Ser. Nos. 09/359,300 and 09/359,304, both incorporated herein by reference.

III. Immobilization and Assembly of Viral Nucleic Acids

The instant invention features a method for constructing a viral nucleic acid, comprising the steps of immobilizing one fragment of a double-stranded DNA sequence on a solid support and assembling this fragment with another fragment of the double-stranded DNA sequence until the double-stranded DNA sequence is fully assembled. In the instances where the infectious viral nucleic acid is DNA, the assembled viral nucleic acid may be used directly after cleavage from the solid support. In the instances where the infectious viral nucleic acid is RNA, an additional in vitro transcription step may be required to convert the assembled double-stranded DNA sequence into RNA viral nucleic acid.

A. DNA Immobilization

One of skill in the art will appreciate that there are many ways of immobilizing DNA sequences directly on a solid support (covalently or noncovalently), anchoring DNA sequences to a linker moiety on a solid support. These methods are well taught in the art of solid phase DNA synthesis (Oligonucleotide Synthesis, Ed. M. J. Gait, Oxford University Press (1990) and Protocols for oligonucleotides and analogs; synthesis and properties, Methods Mol. Biol. 20 (1993)). The immobilization methods generally fall into one of the two categories: spotting of presynthesized DNA and in situ synthesis of DNA.

In the first category, solutions containing preprepared DNA are deposited onto known finite areas on a solid support. For example, traditional solid phase DNA synthesis on controlled-pore glass may be employed and then simply printing presynthesized DNA onto the solid support using direct touch or fine micropipetting. DNA may be synthesized on an automated DNA synthesizer, for example, on an Applied Biosystems synthesizer using 5-dimethoxytritylnucleoside □-cyanoethyl phosphoramidites. Synthesis of relatively long DNA sequences may be achieved by PCR-based methods for economical advantages. DNA may be purified by gel electrophoresis, HPLC, or other suitable methods known in the art before spotted or deposited on the solid support. Typically, solid supports are overlaid with a positively charges coating, such as amino silane or polylysine and presynthesized probes are then printed directly onto the solid surface. Printing may be accomplished by direct surface contact between the printing reagents and a delivery mechanism. Such delivery mechanism may contain the use of tweezers, pins or capillaries, among others that serve to transfer DNA or reagents to the surface. A variation of this simple printing approach is the use of controlled electric fields to immobilize prefabricated charged DNA to microelectrodes on the array (e.g. WO 99/06593). For example, biotinylated DNA may be directed to individual spots by polarizing the charge at that spot and then anchored in place via a steptavidin-containing permeation layer that covers the surface. Some of the advantages of spotting technologies include ease of prototyping and therefore rapid implementation, low cost and versatility.

In the second category, DNA are prepared by in situ synthesis on the solid support in a step-wise fashion. With each round of synthesis, nucleotides are added to growing chains until the desired length is achieved. In general, in situ DNA synthesis on a solid support may be achieved by two general approaches. First, photolithography may be used to fabricate DNA on the solid support. For example, a mercury lamp is shone through a photolithograhic mask onto the chip surface, which removes a photoactive group, resulting in a 5′ hydroxy group capable of reacting with another nucleoside. The mask therefore predetermines which nucleotides are activated. Successive rounds of deprotection and chemistry result in DNA with increasing length. This method is disclosed in, e.g., U.S. Pat. Nos. 5,143,854, 5,489,678, 5,412,087, 5,744,305, 5,889,165, and 5,571,639.

The second approach is the “drop-on-demand” method, which uses technology analogous to that currently employed in “ink-jet” printers (Schena et al., TIBTECH, 16:301-306 (1998)). This approach typically utilizes piezoelectric or other forms of propulsion to transfer reagents from miniature nozzles to solid surfaces. For example, in the case of solid phase DNA synthesis, the printer “head” travels across the solid support, and at each spot, electric current expands an adapter, encircling a tube containing reagents for one of the four DNA bases, forcing a microliter drop of reagents onto the coated surface. Following washing and deprotection, the next cycle of DNA synthesis is carried out.

If single stranded polynucleotides are immobilized on an array, they may be converted to a double-stranded polynucleotide array. There are many ways to prepare double-stranded DNA polynucleotide arrays. One method of preparation is simply using primers, polymerase, and dNTPs to make double stranded polynucleotide array. Another method is by hybridizing the single-stranded immobilized polynucleotide with a double-stranded polynucleotide containing a complementary single-stranded end, followed by treatment with DNA ligase, which results in double-stranded polynucleotides. This method is described in DeRisi et al., Science 278:680-686 (1997) and Braun et al., Nature 391:775-778 (1998). Another method of preparing double-stranded polynucleotide arrays by synthesizing a constant sequence at every position on an array and then annealing and enzymatically extending a complementary primer is described in PCT publication WO 99/07888 and Bulyk et al. Nature Biotechnology, 17:573-577 (1999).

Solid support for use in the instant invention include cellulose, nitrocellulose, nylon membranes, controlled-pore glass beads, acrylamide gels, polystyrene matrices, activated dextran, avidin/streptavidin-coated polystyrene beads, agarose, polyethylene, functionalized plastic, glass, silicon, aluminum, steel, iron, copper, nickel and gold. Some solid supports, such as wafers of aluminum, steel, iron, copper, nickel, gold, and silicon may require functionalization prior to attachment of DNA. Any of a number of methods commonly employed in the art may be utilized to immobilize DNA on a solid support. These methods, including methods for cleaving DNA from solid support, are summarized in U.S. Pat. 5,700,642.

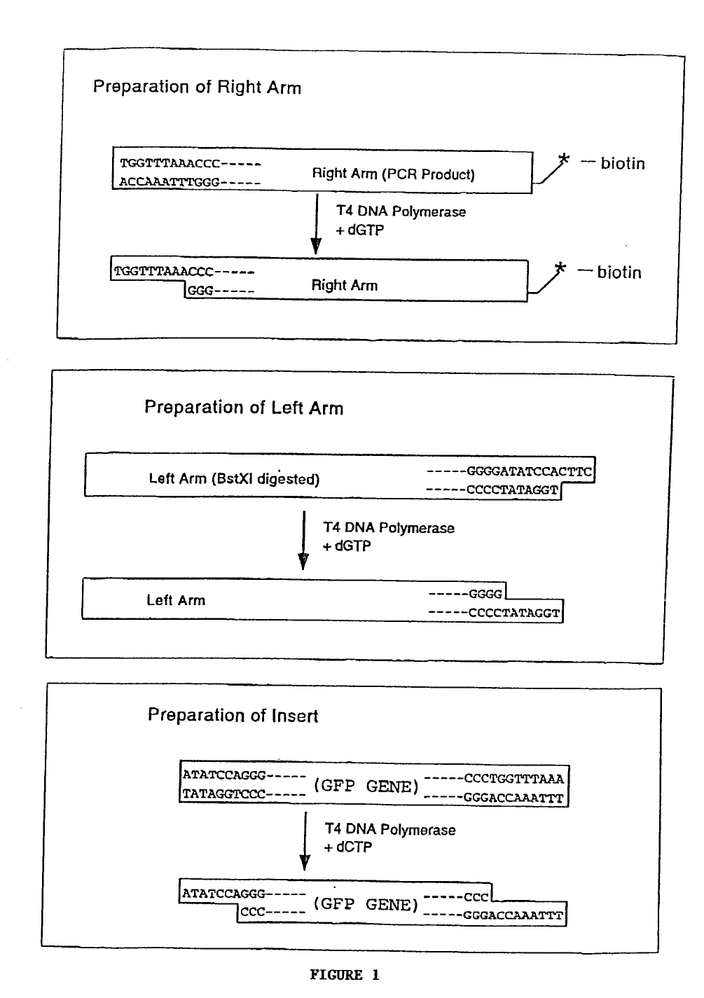

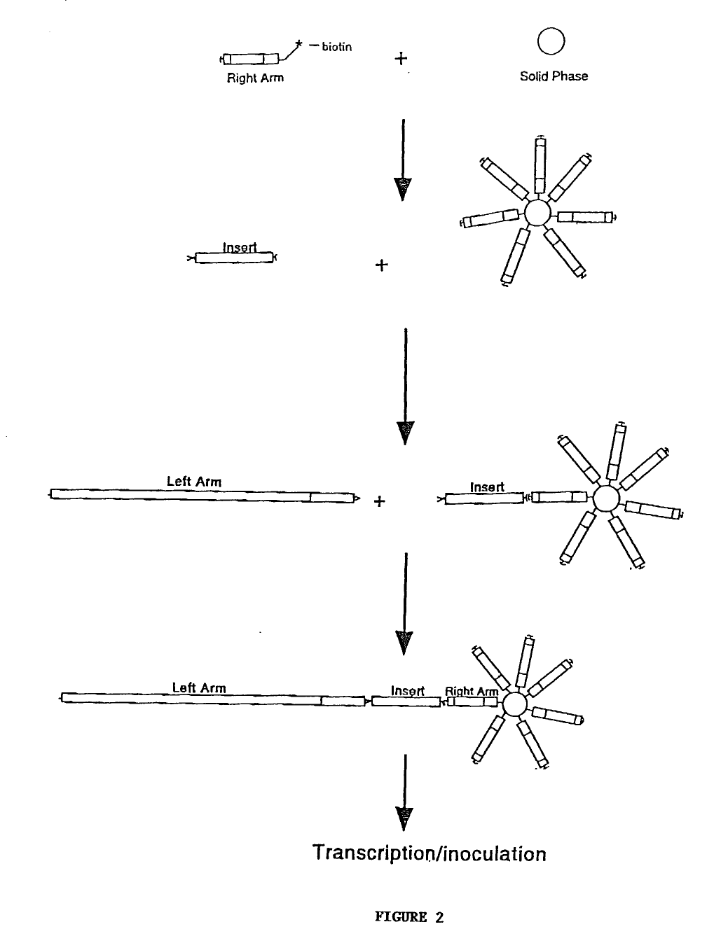

Of course, immobilized DNA on a solid support may be purchased from commercial vendors. For example, biotinylated DNA and streptavidin or avidin-coated solid supports are commercially available (e.g., Promega, Madison, Wis.). In preferred embodiments of the instant invention, the biotinylated DNA for use in immobilization to streptavidin or avidin-coated solid supports are used (see FIGS. 1 and 2). A variety of biotinylation reagents are commercially available (e.g., Promega) which are functionalized to react with DNA or modification thereof. Typically, a DNA is biotinylated by incorporation of biotinylated dNTP containing an intervening spacer arm. The biotinylated DNA is then immobilized by attachment to a streptavidin-coated support. Due to the strong non-covalent biotin/streptavidin interaction, the immobilized DNA is considered to be essentially irreversibly bound to the solid support. The resulting immobilized complex is unaffected by most extremes of pH, organic solvents, and other denaturing agents. An alternative to avidin(streptavidin)-biotin immobilization is incorporation of a digoxigenin molecule (Sigma, St. Louis, Mo.) in the modified DNA with subsequent capture using anti-digoxigenin antibodies.

Enzymatic methods may also be utilized for coupling a DNA to a solid support (U.S. Pat. 5,700,642). In one exemplary embodiment, a poly(dA) tail is added to the 3′ ends of a double-stranded DNA using 3′ terminal transferase. The (dA)-tailed DNA is then hybridized to oligo(dT)-cellulose. To covalently link DNA to the solid support, the hybridized complex is first reacted with a Klenow fragment of DNA polymerase I, followed by treatment with T4 DNA ligase. The unligated strand of DNA is separated from the immobilized strand by heating followed by extensive washing. The method results in single-stranded DNA covalently linked by its 5′ end to a solid support.

It will be appreciated by one of skill in the art that DNA may be labeled directly or indirectly by any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³²P, or ³³P), enzymes (e.g., horse radish peroxidase, alkaline phosphatase and others commonly used in an ELISA), colorimetric labels such as colloidal gold or colored glass or plastic (e.g., polystyrene, polypropylene, latex, etc.) beads.

Means of detecting such labels are well known to those of skill in the art. For example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

B. Assembly of Viral Nucleic Acids

One skilled in the art will appreciate that there are many ways to assemble fragments of a double-stranded DNA into its full length. In preferred embodiments of the instant invention, the assembly of DNA fragments is accomplished by first generating complementary single stranded ends for fragments of the double-stranded DNA; hybridizing one fragment with another fragment of the double-stranded DNA sequence, and ligating the two fragments of the double-stranded DNA sequence. This process is repeated until the full length DNA is assembled. The complementary single stranded ends are typically 1 to 15 nucleotides long, e.g. from 2 to 10, or from 4 to 8 nucleotides long.

There are many ways to generate single stranded ends for DNA fragments. In one method, fragments of the double-stranded DNA are first digested with a restriction endonuclease enzyme which recognizes specific sequences within the fragments. The ends of the restriction endonuclease-treated DNA fragments are further manipulated, if necessary, to make them compatible for annealing and ligation. DNA ligase is then added to the mixture, ligating the DNA fragments together (Sambrook et al. (2nd ed.), Cold Spring Harbor Laboratory, Cold Spring Harbor (1989); Methods in Enzymol. (Vols. 68, 100, 101, 118, and 152-155) (1979, 1983, 1986 and 1987)).

In another method, the template-independent terminal transferase activity of a DNA polymerase, such as Taq polymerase is exploited. The terminal transferase adds a single adenosine nucleotide to the 3′ ends of the DNA fragment, thus generating a one-nucleotide sticky end (Kovalic et al., Nucleic Acids Res. 19:4560 (1991)).

Another approach for generating complementary ends for DNA fragments is splice overlap extension (SOE) PCR (Horten et al., Gene 77:61-68 (1989) and Yon et al., Nucleic Acids Res. 17:4895 (1989)). The primers are designed so that the ends of the PCR products contain complementary sequences. When these PCR products are mixed, denatured, and reannealed, the strands having the matching sequences at their 3′ ends overlap and act as primers for each other. Extension of this overlap by DNA polymerase produces a molecule in which the original sequences are spliced together.

An alternative way to produce single-stranded ends employs uracil DNA glycosylase (Buchman et al., Focus 14:41-45 (1992)). This enzyme cleaves all dUMPs which are incorporated into the PCR primers. The selective removal of dUMPs by uracil DNA glycosylase generates single-stranded 3′ overhangs in the PCR products, which are then annealed to another fragment with complementary single-stranded ends.

Another method for generating single-stranded ends is described in Padgett et al., Gene, 168:31-35 (1996). This technique employs PCR combined with the capacity of the type-IIS restriction endonuclease (ENase) Eam 1104I to cut outside its recognition sequence. Primers that contain the Eam 1104I recognition site (5′CTCTTC) are used to amplify the DNA fragments. Because the ENase is inhibited by site-specific methylation in the recognition sequence, all internal Eam 1104I sites present in the DNA can be protected by performing OCR in the presence of 5-methyl-deoxycytosine. The primer-encoded Eam 1104I sites are not affected by the modified nucleotides since the newly synthesized strand does not contain any cytosine residues in the recognition sequence. In addition, the ENase's ability to cleave several bases downstream from its recognition site allows the removal of superfluous, terminal sequences from the amplified DNA fragments, resulting in 5′ overhangs that are defined by the nucleotides present within the cleavage site. Thus, the elimination of extraneous nucleotides and the generation of unique, non-palindromic sticky ends permits the specific hybridization between DNA fragments with complementary ends during the subsequent ligation event.

In preferred embodiments of the instant invention, an enzyme with exonuclease activity is employed to generate single stranded ends for DNA fragments. Trimming of DNA fragments with the 3′-5′ exonuclease activity in the presence of suitable deoxynucleotides results in fragments containing single stranded extensions. Exonuclease activity of various DNA polymerases such as T4 or T7 DNA polymerases, Pfu polymerase, the Klenow fragment of DNA polymerase I, and Deep Vent DNA polymerase among others may be used. The activity of these exonucleases can be modulated, for instance, by shifting off the optimal pH and/or temperature range or by adding reagents to the reaction mixture. The exonuclease activity can also be modulated by way of functional groups, such as at the C-2′ position of the sugar moiety of the nucleotide building block or at the phosphodiester bond.

In particularly preferred embodiments, the 3′-5′ exonuclease activity of T4 DNA polymerase is utilized (see FIG. 1). The 3′-5′ exonuclease activity of T4 DNA polymerase have previously been used in ligation-independent cloning (Aslanidis et al., PCR Methods Appl. 4:172-177 (1994); Yang et al., Nucleic Acids Res. 21:1889-1893 (1993); Dietmaier et al., Nucleic Acids Res. 21:3603-3604 (1993); and Cease et al., Biotechniques, 14:250-255 (1993)). T4 DNA polymerase may be used in combination with a predetermined dNTP to specifically remove nucleotides from each 3′ end of DNA fragments, providing fragments with 5′-extending single-stranded ends of defined sequence and length. This method does not require restriction enzymes.

In one embodiment of the instant invention, the replication-compatible nucleic acid sequences are divided into two “arms”: the left arm and the right arm (see FIG. 2). Each arm may encode one or more proteins or regulatory sequence for expressing an insert nucleic acid sequence that is sandwiched between the left and right arms after the assembly. The insert may be derived from PCR product, cDNA reaction, restriction digest, or other heterologous nucleic acid mixtures. The insert may contain sequences expressing RNAs, proteins, or peptides of interest. The left and right arms may each have separate asymmetric overhangs that permit the two arms to be brought together by the intervening inert, which has termini compatible with both the left and right arms. The overhanging ends of the left and right arms are non-palindromic, and therefore not self-compatible. The termini of the left arm, right arm and insert are such that the ligation of the left and right arms to the insert ensures the assembly into a proper configuration to yield infectious viral transcripts. The sequence contained in the insert may then be in a correct orientation and genomic position to permit its expression from the virus in host cells.

In a preferred embodiment of this invention, the left arm of this system may encode a replicase or fragments thereof and a movement protein or fragments thereof. The right arm may encode a coat protein or fragments thereof, a 3′ untranslated region (3′-NTR) or fragments thereof, and a ribozyme sequence or fragments thereof. The replicase, movement protein, coat protein, 3′-NTR, and ribozyme sequence may be native to each other, i.e. they are derived from the same viral source. Alternatively, the left or right arm may each be a hybrid containing two or more viral sources. The left and right arms may additionally contain, either from sources native to or non-native sources, promoter sequences, internal initiation sites, one or more packaging signals, 5′ NTRs, among others. In a particularly preferred aspect of this embodiment, the left arm may contain nucleic acid sequences encoding, from 5′ to 3′, a T7 RNA polymerase promoter, a replicase from a viral source, a movement protein from a viral source, and one or more subgenomic promoters that control the expression of the insert sequence. The right arm may contain sequences encoding, from 5′ to 3′, one or more sequences that control the expression of the coat protein, a coat protein from a viral source, a viral 3′ NTR, and a ribozyme sequence for generating the desired 3′ terminus on the transcribed molecules.

In another embodiment of the instant invention, the right arm may be synthesized by PCR based methods and may have a biotin group incorporated into the reverse (3′) primer. The resulting biotinylated PCR product representing the right arm may then be immobilized upon streptavidin paramagnetic beads. Treatment of the DNA with T4 DNA polymerase and a single dNTP, for example, dGTP, may give a 5′ overhang as a result of the exonuclease activity of the polymerase. The insert DNA may be treated with T4 DNA polymerase with a single dNTP to generate 5′ overhangs on its termini, the 3′ of which is compatible with the 5′ of the right arm. The 5′ terminus of the insert DNA is compatible with the 3′ terminus of the left arm, which may be generated similarly. The ligation reactions in the assembly of the virus on the paramagnetic beads may be carried out sequentially, with the insert being ligated to the immobilized right arm first, followed by washing of the bead complex and then ligation of the left arm. Following the subsequent wash, in vitro transcription will be carried out to generate infectious RNA transcripts.

In addition to using double-stranded DNA in the assembly process, one can join single-stranded DNA molecules to construct a full length virus-encoding molecule. In essence, after immobilization of minus sense single stranded DNA, (right arm) at the 5′ end, a minus-sense single stranded insert DNA is added along with a DNA molecule of the opposite sense that overlaps in part with both the insert and right arms to bring them into juxtaposition with one another at their 5′ and 3′ termini, respectively, DNA ligase added to the reaction will catalyze the joining of the insert and right arm strands. Similarly, the left arm can be ligated onto the insert-right arm assembly. After construction of the full-length molecule, an oligonucleotide complimentary to the T7 RNA polymerase promoter is annealed followed by in vitro transcription. Basically, this is a long single stranded DNA molecule that has been pieced together, the double stranded region at the T7 promoter is required to initiate transcription which can then proceed using the single-stranded template.

IV. Delivery of Viral Nucleic Acids in a Host and Selection of Products of Interest

The delivery of the viral nucleic acids constructed in a cell-free manner is similar to those constructed by cloning in bacterial cells. These methods are well known in the art. For example, the plant viral nucleic acids' entrance into plant hosts may be affected by the inoculation of in vitro transcribed RNA, inoculation of virions, or internal inoculation of plant cells from nuclear cDNA, or the systemic infection resulting from any of these procedures. In all cases, the co-infection may lead to a rapid and pervasive systemic expression of the insert nucleic acid sequence in plant cells. The systemic infection of the plant by the insert sequence may be followed by the growth of the infected host to produce the desired product, and the isolation and purification of the desired product, if necessary. The growth of the infected host is in accordance with conventional techniques, as is the isolation and the purification of the resultant products.

After a plant host is infected with a library or individual clones of sequence variants generated in a cell-free manner, one or more desired traits are screened and selected. The desired traits may include biochemical or phenotypic traits. Phenotypic traits may include, but not limited to, host range, viral infectivity, tolerance to herbicides, tolerance to extremes of heat or cold, drought, salinity or osmotic stress; resistance to pests (insects, nematodes or arachnids) or diseases (fungal, bacterial or viral); male or female sterility; dwarfness; early maturity; improved yield, vigor, heterosis, nutritional qualities, flavor or processing properties, and the like. Biochemical traits may be related to, for example, the promoter activities, replication activities, translational activities, regulatory activities, movement activities (local and systemic), signaling pathway, extraction/purification properties, etc. The screening of sequence libraries is typically followed by rescue of the viruses from populations conferring desired traits by PCR and if necessary, re-screening of sub-libraries in secondary screens. In some embodiments, sequences of the viral nucleic acids conferring desired traits may be determined and compared with the template sequences.

A detailed discussion of methods for expressing viral nucleic acid in a host and selecting a desired trait in a host is presented in two co-pending and co-owned U.S. patent application Ser. Nos. 09/359,300 and 09/359,304, both incorporated herein by reference.

In order to provide a clear and consistent understanding of the specification and the claims, including the scope given herein to such terms, the following definitions are given:

5′ or 3′ NTR: nontranslated region of a viral genome at the 5′ or 3′ end, typically longer than 25 nucleotides and shorter than 500 nucleotides.

Cis-acting (cis-dependent): interaction of a molecule or complex with itself or between a gene product with the nucleic acid from which it was expressed.

Coat protein (capsid protein): an outer structural protein of a virus.

Gene: a discrete nucleic acid sequence responsible for a discrete cellular product.

Host: a cell, tissue or organism capable of replicating a vector or viral nucleic acid and which is capable of being infected by a virus containing the viral vector or viral nucleic acid. This term is intended to include prokaryotic and eukaryotic cells, organs, tissues or organisms, or in vitro extracts thereof, where appropriate.

Infection: the ability of a virus to transfer its nucleic acid to a host or introduce viral nucleic acid into a host, wherein the viral nucleic acid is replicated, viral proteins are synthesized, and new viral particles assembled.

Internal initiation site: any of the internal regions that direct ribosome-mediated translation of mRNA into polypeptides.

Movement protein: a noncapsid protein required for cell-to-cell movement of RNA replicons or viruses in plants.

Non-native (foreign): any sequence that does not normally occur in the virus or its host.

Open Reading Frame: a nucleotide sequence of suitable length in which there are no stop codons.

Packaging signal: the RNA sequence(s) responsible for enclosing the RNA within the capsid or coat protein(s) to form a mature virion.

Plant Cell: the structural and physiological unit of plants, consisting of a protoplast and the cell wall.

Plant Tissue: any tissue of a plant in planta or in culture. This term is intended to include a whole plant, plant cell, plant organ, protoplast, cell culture, or any group of plant cells organized into a structural and functional unit.

Promoter: the 5′-flanking, non-coding sequence adjacent to a coding sequence which is involved in the initiation of transcription of the coding sequence.

Protoplast: an isolated cell without cell walls, having the potency for regeneration into cell culture or a whole host.

PCR: a broad range of polynucleotide amplification techniques for increasing the number of copies of specific polynucleotide sequences. Examples of polynucleotide amplification reactions include, but not limited to, polymerase chain reaction (PCR, U.S. Pat. Nos. 4,683,202 and 4,683,195), nucleic acid sequence based amplification (NASB), self-sustained sequence replication (3SR), strand displacement activation (SDA), ligase chain reaction (LCR), rolling-circle amplification (RCA), Qβ replicase system, and the like (Isaksson and Landegren, Curr. Opin. Biotechnol. 10:11-15 (1999); Landegren, Curr. Opin. Biotechnol. 7:95-97 (1996); and Abramson et al., Curr. Opin. Biotechnol. 4:41-47 (1993)).

Solid support: a material or a group of materials having a rigid or semi-rigid surface or surfaces. Solid support includes, but not limited to, cellulose, nitrocellulose, nylon membranes, controlled-pore glass beads, acrylamide gels, polystyrene matrices, activated dextran, avidin/streptavidin-coated polystyrene beads, agarose, polyethylene, functionalized plastic, glass, silicon, aluminum, steel, iron, copper, nickel, gold, and the like.

Subgenomic mRNA promoter: a promoter that directs the synthesis of an mRNA smaller than the full-length genome in size.

Trans-acting: interaction of a molecule or complex on other molecule(s) independent from itself or independent from the nucleic acid from which it was expressed.

Vector: a self-replicating nucleic acid molecule that contains non-native sequences and which transfers nucleic acids between cells.

Virion: a particle composed of viral nucleic acid, viral coat protein (or capsid protein).

Virus: an infectious agent composed of a nucleic acid encapsulated in a protein.

### EXAMPLES OF THE PREFERRED EMBODIMENTS

The following examples further illustrate the present invention. These examples are intended merely to be illustrative of the present invention and are not to be construed as being limiting.

### Example 1

In this example, replication-competent viruses expressing a foreign gene were constructed in a cell-free manner. More specifically, the gene encoding the green fluorescent protein (GFP) from Aquorea victoria was used as a marker to demonstrate infection and replication by virus that had been assembled from discrete DNA fragments in a cell free manner.

The right arm, encoding all viral sequences 3′ from where the foreign gene was to be inserted into the viral genome, was generated using standard methods of PCR. The 3′ (reverse) PCR primer was synthesized with a biotin group covalently attached at its 5′ terminus and was paired with the appropriate 5′ (forward) primer for amplification of a 1200 bp fragment encoding the 3′ terminal sequences of the tobamovirus genome located in the plasmid, p30B-5XPL, a derivative of p30B, an infectious full-length tobamovirus clone (Shivprasad et al, Virology 255:313-323 (1999)). The biotinylated PCR product was then gel-purified and a 3 μg aliquot was treated with 3 units of phage T4 DNA polymerase (Novagen; Madison, Wis.) in 50 mM Tris-HCl (pH 8.0), 10 mM MgCl₂, 50 μg/ml bovine serum albumen, 5 mM DTT and 2.5 mM dGTP for 20 minutes at 37° C. to generate a nine base 5′ overhang at its 5′ terminus. Following this step, the DNA was purified using a Strataprep DNA purification column (Stratagene; La Jolla, Calif.) and immobilized by incubation with 50 μg of MagneSphere streptavidin-coated paramagnetic beads (Promega; Madison, Wis.) at 50° C. in the presence of 4.5 M NaCl, 10 mM Tris-HCl (pH 8.0), and 1 mM EDTA. After immobilization of the DNA, the DNA bead complexes were washed several times by sedimentation in a magnetic field after suspension in 10 mM Tris-HCl pH 8.0 and 1 mM EDTA supplemented with 0.01% Tween 20 to prevent clumping.

The left arm contains a T7 RNA polymerase promoter followed by all the viral genomic sequences 5′ to the insertion point of the foreign gene. This DNA was derived from p30B-5XPL after digestion with the restriction enzymes BstXI and PstI and treatment with T4 DNA polymerase as described above.

The GFP gene insert used in this example was generated by PCR using the GFP gene as template. The PCR primers was designed to anneal to the 5′ and 3′ extremities of the GFP open reading frame. The 5′ ends of the primers were engineered to render 5′ overhangs upon treatment with T4 DNA polymerase as described above, with the single exception that dCTP was used, instead. The resulting overhangs on the 5′ and 3′ ends of the treated PCR product were compatible for ligation with the overhangs on the right and left arms, respectively.

Assembly of the GFP-containing viral sequences was performed by sequential ligation of the various components into the desired conformation. First, a molar excess of prepared GFP PCR product was mixed with the above-described right arm-magnetic bead complexes in the presence of T4 DNA ligase with a PEG-containing ligase buffer (Gibco-LTI). After extensive washing with 10 mM Tris-HCl pH 8.0, 1 mM EDTA and 0.01% Tween 20, the resultant DNA molecules were analyzed by digestion of a portion of the DNA:bead complexes with the restriction enzyme HinDIII followed by agarose gel analysis to confirm that the desired ligation event had occurred. Next, the above-described left arm preparation was mixed with the GFP-right arm:magnetic bead complexes and ligated as above. An aliquot was removed and analyzed by restriction digestion to confirm that the desired ligation product had resulted. After confirmation that the viral sequences had been assembled properly with the GFP gene sequence in the appropriate location and orientation, the DNA:bead complexes were washed extensively as described above and used as template in vitro transcription using a phage T7 RNA polymerase reaction kit (Ambion, Austin, Tex.). The resulting capped, infectious RNA transcripts were then introduced into protoplasts of tobacco BY-2 suspension culture cells by electroporation as described by Wantanabe et al., Virology 133:18-24 (1987).

At 12-18 hours after protoplast infection, fluorescence emitted by the GFP encoded by the virus clone was observed in a majority of the cells confirming that the RNA transcript derived from the DNA:bead complexes was infectious and hence, that the sequentially assembled virus-encoding DNA molecules had been assembled in the desired configuration so as to permit virus replication and expression of the inserted foreign gene sequences.

### Example 2

This example demonstrates the cell-free construction of libraries of nucleic acid sequence expressing a large number of specific genes. Nucleic acids from a particular microbial organism whose genome has been sequenced are prepared to represent all known open reading frames. Gene-specific primer pairs are prepared for all genes. Each primer pairs are constructed to be amenable to T4 polymerase treatment to generate the desired cohesive termini for ligation to virus arms. In 96 or 384-well format, each of those open reading frames is amplified by PCR. After purification of the PCR products and generation of the desired overhangs by T4 DNA polymerase, each heterologous gene sequence in arrayed format is ligated to the immobilized right arm, washed, and then ligated to left arm and transcribed in vitro to generate an ordered array of infectious viral nucleic acid sequences, each encoding a different ORF for expression in vivo. Each resulting virus would express a different heterologous gene.

### Example 3

This example shows the production of very large and diverse libraries of sequences. This library is a pool of virus particles, each containing a different heterologous sequence. The sequences could encode cDNAs, DNAs, random libraries of peptides, RNA aptamers, ribozyme sequences, or gene fragments. Such a library contains as many as 100,000,000 different sequences. The basic methodology is based on a one-tube method in which the desired insert sequences, derived from random oligonucleotides, PCR products, cDNA, or genomic DNA fragments modified to have the appropriate overhangs are ligated to the immobilized right arm of the virus. After washing, the left arm of the virus is ligated to the insert:right arm assemblies which will then be washed again. The reconstructed virus-encoding DNAs are then be used as template for in vitro transcription. Infectious RNA transcripts are then introduced into large numbers of plant protoplasts by standard means such as electroporation or PEG-mediated transfection. After the appropriate time in culture, the cells are harvested and lysed, and the liberated virus purified. The virus preparation will be the library that can then be re-introduced into cells for functional screening or selection.

Although the foregoing invention has been described in some detail by way of illustration and example for purposes of clarity of understanding, it will be obvious that certain changes and modifications may be practiced within the scope of the appended claims.

All publications, patents, patent applications are herein incorporated by reference in their entirety to the same extent as if each individual publication, patent, or patent application was specifically and individually indicated to be incorporated by reference in its entirety.

1. A method for constructing a viral nucleic acid in a cell-free manner, comprising the steps of:
(a) immobilizing a presynthesized first fragment of a double-stranded DNA sequence, which corresponds to a viral nucleic acid sequence, directly on a solid support;
(b) treating said first fragment and a second fragment of said double-stranded DNA sequence with an enzyme having 3′-5′ exonuclease activity, which provides said first fragment and said second fragment each with 5′-extending single-stranded end of defined sequence and length, wherein said single-stranded ends of first and second fragments are complementary to each other;
(c) assembling by hybridization and ligation of said first fragment with said second fragment of said double-stranded DNA sequence ; and
(d) treating and assembling said second fragment and a subsequent fragment according to steps (b) and (c); and
(e) repeating step (d) with subsequent fragments until the double-stranded DNA sequence is fully assembled.

2. The method according to claim 1, wherein said enzyme is T4 DNA polymerase.

3. The method according to claim 1, wherein said solid support is a streptavidin-coated solid support and said first fragment of the double-stranded DNA sequence is biotinylated.

4. The method according to claim 1, wherein said first fragment immobilized on the solid support corresponds to the 3′ portion of said viral nucleic acid.

5. The method according to claim 1, wherein said viral nucleic acid is native to an RNA plant virus.

6. The method according to claim 9, wherein said viral nucleic acid is native to a single-stranded, plus sense RNA plant virus.

7. The method according to claim 1, wherein said viral nucleic acid is native to an animal virus.

8. The method according to claim 1, wherein said viral nucleic acid contains one or more sequences non-native to said viral nucleic acid.

9. The method according to claim 1, wherein said viral nucleic acid contains one or more non-native promoters.

10. The method according to claim 9, wherein said viral nucleic acid contains a non-native sequence fused with a native sequence encoding a coat protein or fragments thereof.

11. The method according to claim 12, wherein said non-native sequence encodes a product selected from the group consisting of enzymes, antibodies, hormones, pharmaceuticals, vaccines, pigments, and antimicrobial polypeptides.

12. The method according to claim 12, wherein said non-native sequence is a regulatory sequence.

13. The method according to claim 1, wherein said method is used for high throughput construction of viral nucleic acids.


SEQUENCE LISTING (12 sequences)

SEQ ID NO: 1 - DNA, 13 bp, Artificial Sequence
  Right arm (PCR product)
atggtttaaaccc

SEQ ID NO: 2 - DNA, 12 bp, Artificial Sequence
  Right Arm (PCR Product)
accaaatttggg

SEQ ID NO: 3 - DNA, 12 bp, Artificial Sequence
  Right Arm
tggtttaaaccc

SEQ ID NO: 4 - DNA, 15 bp, Artificial Sequence
  Left Arm (BstXI digested)
ggggatatccacttc

SEQ ID NO: 5 - DNA, 11 bp, Artificial Sequence
  Left Arm (BstXI digested)
cccctataggt

SEQ ID NO: 6 - DNA, 12 bp, Artificial Sequence
  Left arm
cccctastaggt

SEQ ID NO: 7 - DNA, 10 bp, Artificial Sequence
  (GFP Gene)
atatccaggg

SEQ ID NO: 8 - DNA, 11 bp, Artificial Sequence
  (GFP Gene)
ctataggtccc

SEQ ID NO: 9 - DNA, 12 bp, Artificial Sequence
  (GFP Gene)
ccctggtttaaa

SEQ ID NO: 10 - DNA, 12 bp, Artificial Sequence
  (GFP Gene)
gggaccaaattt

SEQ ID NO: 11 - DNA, 10 bp, Artificial Sequence
  (GFP Gene)
atatggaggg

SEQ ID NO: 12 - DNA, 12 bp, Artificial Sequence
  (GFP Gene)
gggaccaaattt